(19) United States
(12) Patent Application Publication
(10) Pub. No.: US 2018/0256505 A1
(43) Pub. Date: Sep. 13, 2018

(54) PROBIOTIC CAPSULE AND METHODS OF PREPARING THE SAME

(71) Applicant: The Clorox Company, Oakland, CA (US)

(72) Inventors: William Robert King, Walnut Creek, CA (US); Vidya Ananth, Livermore, CA (US); John Eleftheriou Theofanous, Tarpon Springs, FL (US); Gregory Thomas Horn, Lighthouse Point, FL (US); Maha Y. El-Sayed, Fremont, CA (US)

(21) Appl. No.: 15/916,878

(22) Filed: Mar. 9, 2018

Related U.S. Application Data
(60) Provisional application No. 62/469,407, filed on Mar. 9, 2017.

(51) Int. Cl. A61K 9/48

(52) U.S. Cl. CPC A61K 9/4816; A61K 2035/115; A61K 9/4875; A61K 9/4866

(57) ABSTRACT

Encapsulated probiotic compositions that deliver a minimum of 5 Billion CFU/capsule in potency and a minimum of at least 1 probiotic strain with clinical efficacy throughout shelf life are disclosed herein. In some embodiments, the encapsulated probiotic compositions meet the USDA certified organic labeling requirement, including 5% or less of non-organic materials.

### CROSS REFERENCE TO RELATED APPLICATIONS

This application claims priority to U.S. Provisional Patent Application No. 62/469,407 filed on Mar. 9, 2017, which is hereby incorporated by reference in its entirety.

### BACKGROUND OF THE DISCLOSURE

The present disclosure relates generally to encapsulated probiotic compositions that deliver a minimum of 5 Billion CFU/capsule in potency and a minimum of at least 1 probiotic strain with clinical efficacy throughout shelf life, as well as to methods of preparing the compositions. It is particularly suitable for the compositions to have water activity (a_{w}) levels such as to prevent undesirable brittleness of the encapsulated probiotic composition, while providing improved shelf-life stability. In particularly suitable embodiments, the encapsulated probiotic compositions meet the USDA certified organic labeling requirement, including 5% or less of non-organic materials.

While probiotics are generally not covered under USDA organic rules, organic foods have grown in popularity such that it would be commercially desirable to provide an organic probiotic composition. Previous efforts to commercialize an organic probiotic composition in capsule form have been unsuccessful for a variety of reasons: 1) unavailability of organic capsule technology; 2) inability to obtain organic certification for cultures due to regulatory standards of organic cultures for probiotics; 3) availability of relevant organic excipients that complement or enhance probiotic functionality; and 4) the ability to combine all of these ingredients into an efficacious product that is stable during distribution and shelf life required for a commercial product.

As probiotics are not considered an agricultural commodity and cannot be organically certified according to USDA guidelines, organic probiotic composition-containing products can still be made to include non-organic cultures if the other organic components constitute at least 95% of the total product by weight; that is, up to 5% of non-organic culture composition could be used in a product in which all other ingredients meet organic requirements to allow the product to be labeled an organic product under USDA guidelines. Conventionally, however, initial levels of 1 Billion CFU/capsule or less, and typically less than 5 Billion CFU/capsule, is insufficient to provide relevant levels of culture stability to ensure survival of clinically studied potency levels through end of product shelf life. This constraint has severely hampered development of organic probiotics as clinical support for efficacy is an Federal Trade Commission (FTC) defined standard for truth in advertising and labeling of probiotic supplements.

Furthermore, organic capsules may require very high moisture levels to maintain structural integrity, typically more than twice the level of water activity (a_{w}) as traditional probiotic capsules. High moisture allows these capsules to meet USDA organic standards (that is, requiring that at least 95% of the overall composition by weight be made of organic material). This high moisture content can, however, create other problems such as reduced shelf-life and probiotic instability as described herein.

Based on the foregoing, there is a need in the art for an encapsulated probiotic composition that delivers a stable, commercially viable probiotic, with efficacious potency through end of shelf life. It would be further advantageous if the encapsulated probiotic composition met USDA organic labeling requirements.

### BRIEF DESCRIPTION

The present disclosure is directed to encapsulated probiotic compositions having water activity (a_{w}) levels such to prevent undesirable brittleness of the capsule, while providing improved shelf-life stability. These encapsulated probiotic compositions can be provided in various levels of potency, and, in some embodiments, the probiotic capsules meet the USDA certified organic labeling requirement, including 5% or less of non-organic materials, while providing a potency higher than conventional probiotic products, and typically greater than 5 Billion CFU/capsule.

In one aspect, the present disclosure is directed to an encapsulated probiotic composition comprising a capsule and 5% or less by weight of the composition of a probiotic. The probiotic composition has a potency of at least 5 Billion CFU/capsule through end of product shelf life.

In another aspect, the present disclosure is directed to an encapsulated probiotic composition comprising a capsule and 5% or less by weight of the composition of a probiotic. The probiotic composition has an initial water activity (a_{w}) less than 0.60.

In yet another aspect, the present disclosure is directed to kits comprising a container and the above described encapsulated probiotic compositions.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts the hygroscopic properties of excipients for use in the encapsulated probiotic compositions of the present disclosure. Incubation is at 25° C. and 60% relative humidity (RH).

FIG. 2A depicts the water activity (a_{w}) of various Lactobacillus strains over a 3-hour period for use in the encapsulated probiotic compositions of the present disclosure. Incubation is at 36° C. and 60% relative humidity (RH).

FIG. 2B depicts the stability of a commercially available Lactobacillus acidophilus strain at two initial water activity levels over a 2-year period at 4° C. and 25° C.

FIG. 3A depicts exemplary water activity (a_{w}) changes in conventional organic probiotic compositions after storage for one month. Storage conditions include 5° C. and 60% relative humidity (RH). As shown, the water activity is not stabilized until after 1 month, and it varies for each type of organic probiotic composition.

FIG. 3B depicts water activity (a_{w}) changes in exemplary encapsulated probiotic compositions of the present disclosure after storage for seven weeks. Storage conditions include 5° C. and 60% relative humidity (RH) with 3 grams of dessicant.

FIG. 3C depicts water activity (a_{w}) changes in empty capsules after storage for one month at various dessicant levels. Storage conditions include 5° C. and 60% relative humidity (RH). 60 capsules in bottle.

FIG. 4 depicts a minimum breakage water activity threshold of 30 capsules at 25° C.

FIG. 5 depicts a minimum breakage water activity threshold of 30 capsules at 5° C.

FIGS. 6A & 6B depict a minimum breakage water activity threshold of 30 capsules including various dessicant levels at 25° C. (FIG. 6A) and 5° C. (FIG. 6B).

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

Generally, the present disclosure is directed to a probiotic composition in capsule form having improved stability and integrity. In some embodiments, the capsule is an organic capsule. Generally, the encapsulated probiotic composition includes a capsule made of a plant-derived water soluble polysaccharide. Suitable plant-derived water soluble polysaccharides include hydrocolloids such as gums and starches derived from, for example, tapioca, acacia, locust bean, and the like, as well as combinations thereof. Gums and starches defined above may or may not be produced by fermentation or enzymatic modification of organic plant material to produce water binding hydrocolloids such as pullulan, zanthan, exopolysaccharides, and the like, and combinations thereof.

In some embodiments, the encapsulated probiotic composition is an organic probiotic composition, as defined by the USDA certified organic labeling requirement (i.e., including 5% or less of non-organic materials (e.g., probiotics, non-organic excipients and non-organic diluents)). Accordingly, the encapsulated probiotic composition includes 5% or less by weight of at least one probiotic strain, including from about 1% by weight to 5% by weight probiotic strain. Suitable probiotic strains include, for example, one or more strains from the genus Lactobacillus (e.g., Lactobacillus rhamnosus, Lactobacillus acidophilus, Lactobacillus casei, Lactobacillus paracasei, Lactobacillus plantarum, Lactobacillus salivarius, Lactobacillus gasseri), one or more strains from the genus Bifidobacterium (e.g., Bifidobacterium lactis, Bifidobacterium bifidum), one or more strains from the genera: Streptococcus, Lactococcus, Enterococcus, Leuconostoc, Akermansia, and like probiotic strains that are sensitive to water activity. In one embodiment, the probiotic includes a combination of one or more strains of Lactobacillus and one or more strains of Bifidobacterium probiotic strains such as shown in the exemplary compositions described in the exemplary formulations of the Examples below.

It has been found that the encapsulated probiotic composition can be prepared to have a potency ranging from low potency (approximately 1 Billion CFU/capsule) to higher potency (approximately 50 Billion CFU/capsule). Unexpectantly, and advantageously, even when prepared as an organic encapsulated probiotic composition, the encapsulated probiotic composition typically has a potency of greater than 5 Billion CFU/capsule, including a potency ranging from about 5 Billion CFU/capsule to about 30 Billion CFU/capsule, and including about 5 Billion CFU/capsule to about 20 Billion CFU/capsule, and including about 5 Billion CFU/capsule to about 10 Billion CFU/capsule through the end of shelf life. In other embodiments, the encapsulated probiotic composition has a potency ranging from about 10 Billion CFU/capsule to about 30 Billion CFU/capsule through the end of shelf life. It should be understood that the potency of the encapsulated probiotic composition should be maintained at the desired ranges through end of shelf-life of the composition; that is, the potency of the encapsulated probiotic compositions should remain within the range of greater than 1 Billion CFU/capsule to 50 Billion CFU/capsule through the end of shelf-life. As used herein, “shelf-life” refers to the period from the point of producing the finished product (i.e., encapsulated probiotic composition), through packaging, shipping and handling, to storage of the packaged product, typically for a period up to 24 months, including a period ranging from about 12 months to about 24 months, and suitably from about 18 months to about 24 months. Typical storage temperatures range from about 0° C. to about 37° C., including about 4° C. to about 25° C. This is surprising as, conventionally, when limiting a probiotic composition to 5% or less of a product, the potency (CFU counts/gram) is limited to levels of 1 Billion CFU/capsule or less at end of shelf-life. Since most clinical proven dose requirements are at levels of more than 5-10 billion CFU/capsule, before the present disclosure, a product that qualifies as organic is usually of insufficient potency to be able to provide clinically relevant levels of culture to ensure efficacy for specific functional benefits.

In addition to the probiotic strains, the encapsulated probiotic composition of the present disclosure includes at least one excipient. For example, in order to meet the 5% non-organic USDA requirement, an encapsulated probiotic composition may contain an excipient in addition to the probiotics at a level of about 95% or more by weight of the composition. Many functional excipients are agricultural in origin, and recognizing this, it is possible to develop functional excipients that meet USDA organic guidelines. Several of these excipient classes complement or enhance the functionality of probiotic cultures, in particular, selected oligosaccharides and fibers can boost the growth and performance of probiotic strains in the gastrointestinal (GI) tract after consumption. In addition, other excipients have been developed which enhance the immune support, regularity, or vaginal health effects of selected probiotic strains. In order to meet the 5% non-organic USDA requirement, a probiotic capsule will need to include organic excipients in addition to the probiotics, at levels of at least 95%, depending on culture and capsule weights. In addition, such excipients may be chosen to enhance and complement the functional benefits of a probiotic. Since probiotics are very sensitive to water activity (a_{w}) and are highly unstable in the presence of many excipients, which are also naturally hygroscopic (i.e., have a tendency to absorb moisture from the air), this results in undesirably high levels of aw.

In some embodiments, the encapsulated probiotic composition includes excipients that have the proven ability to support the growth of one or more of the probiotic strains used in the composition, such as prebiotic oligosaccharides, prebiotic fibers, and combinations thereof. Particularly, suitable excipients in these embodiments include xylo-oligosaccharide (XOS), fructo-oligosaccharide (FOS), galacto-oligosaccharides (GOS), inulin, aranbinoxylan, xylan, polydextrose (PDX), lactitol, pullulan, gentiobiose, and combinations thereof.

In other suitable embodiments, the excipients for use with the encapsulated probiotic composition include, for example, dried fungal fermentates, yeasts, whole fruits, berries, botanicals, extracts, betaglucan, cereals, cellulose and the like, and combinations thereof.

As stated herein, the encapsulated probiotic composition of the present disclosure must achieve a balance of water activity (a_{w}) (also referred to as water activation (a_{w})) of excipients used therein that is low enough for probiotic stability and high enough for capsule integrity. The encapsulated probiotic compositions of the present disclosure are capable of achieving the ideal balance of water activity by using processes including control and treatment of raw ingredient water activity (a_{w}), selection of specific types of desiccant and desiccant levels for use with the probiotics and excipients, selection of packaging types, and managing the internal equilibration of water activity between the raw ingredients, excipients and capsule.

Water activity (a_{w}) represents the ratio of the partial water vapor pressure of a food to a partial water vapor pressure of pure water under the same conditions. Water activity is an important parameter in controlling water migration of multicomponent products. Undesirable changes are often the result of moisture migration in multicomponent foods and supplements. Moisture will migrate from the region of high a_{w} to the region of lower aw, but the rate of migration depends on many factors such as, for example, relative hygroscopicity of the probiotic/excipient composition, capsule and dessicant. Hygroscopicity will be determined by the relative water binding capacity of the various ingredients. Water activity (a_{w}) of water is 1.0. Sample water activity can be determined using water activity equipment and measurement conditions as known in the art (e.g., Rotronic Water Activity Meter: HYGROLAB C1).

To measure water activity (a_{w}) of probiotic powder, for example, approximately 1.5 grams of probiotic powder is added to a sample container and covered until the measurement is taken. The sample container is then inserted into the sample holder or probe cavity after removing the lid of sample container to take the water activity measurements. To measure water activity (a_{w}) of capsules, such as the encapsulated probiotic composition of the present disclosure, empty capsules are placed in sample container. There should be very little gap between each capsule as they are placed in a sample container. The number of capsules analyzed can vary based on capsule size. The sample container is then inserted into the sample holder or probe cavity to take the water activity measurements.

While control of initial a_{w} for both of the probiotic culture and excipient would seem to be readily attainable objectives, in reality it has proven difficult to work with the specific organic excipients without rapid absorption of moisture during the time required for blending and packaging, as well as during the storage and shelf life of finished probiotic capsules (i.e., encapsulated probiotic compositions). Water activity (a_{w}) of excipients also varies on a lot-to-lot basis. The present disclosure describes additional tools that help to adjust and fine tune a_{w} levels from lot-to-lot in finished probiotic capsule products.

The level of water activity that is needed for probiotic stability is ideally between 0.05 a_{w} and 0.15 a_{w} to ensure acceptable culture stability over time. Further, organic capsules (i.e., capsules including 95% or greater of organic ingredients) normally require high water activities of ˜0.3-0.5 a_{w} in order to maintain sufficient tensile strength during encapsulation, bottling, shipping, and storage. Should the water activity drop below these levels, capsules become more brittle and begin to shatter at a high frequency. Thus, there is a gap between the ideal a_{w} for culture stability versus ideal a_{w} for capsule integrity. Narrowing this gap has been a critical component for enabling production of the encapsulated probiotic compositions of the present disclosure. As such, the encapsulated probiotic compositions of the present disclosure suitably have an initial water activity (a_{w}) at a temperature ranging from about 4° C. to about 37° C., including a temperature of about 4° C. to about 25° C., of less than 0.60, and more suitably, an initial water activity of from about 0.20 to less than 0.60, and more suitably, an initial water activity of from about 0.30 to less than 0.60, and even more suitably, less than 0.30.

In embodiments of the present disclosure in which excipients are included in the encapsulated probiotic composition, it should be understood that the excipients have an initial water activity (a_{w}) of less than 0.30, and suitably, from about 0.10 to about 0.20, to ensure that the resulting encapsulated probiotic compositions have the desired water activity (a_{w}) at all time points from blending and packaging through storage and shelf life.

To adjust and fine tune a_{w} levels, a process for combining these ingredients and maintaining stability is needed. Generally, the present disclosure additionally provides a process for controlling aw, the process including the steps of: calculating the amounts of probiotics and excipients required for encapsulation within the capsule in accordance with a desired dosage; blending the probiotics and excipients to form a bulk composition with a desired initial aw; encapsulating the bulk probiotic composition and measuring aw; filling a container (e.g., bottle) with the encapsulated probiotic composition; adding an effective amount of desiccant to the container in accordance with the initial aw; and equilibrating the contained product at a controlled rate by controlling temperatures, dessicant type and level, and package moisture vapor transfer rates (MVTR) to reach the desired aw. The dessicant could be in the form of a pillow, canister or could be dessicant layered bottle. Suitable dessicant types include, for example, silica gel, calcium oxide, molecular sieves, or a combination thereof.

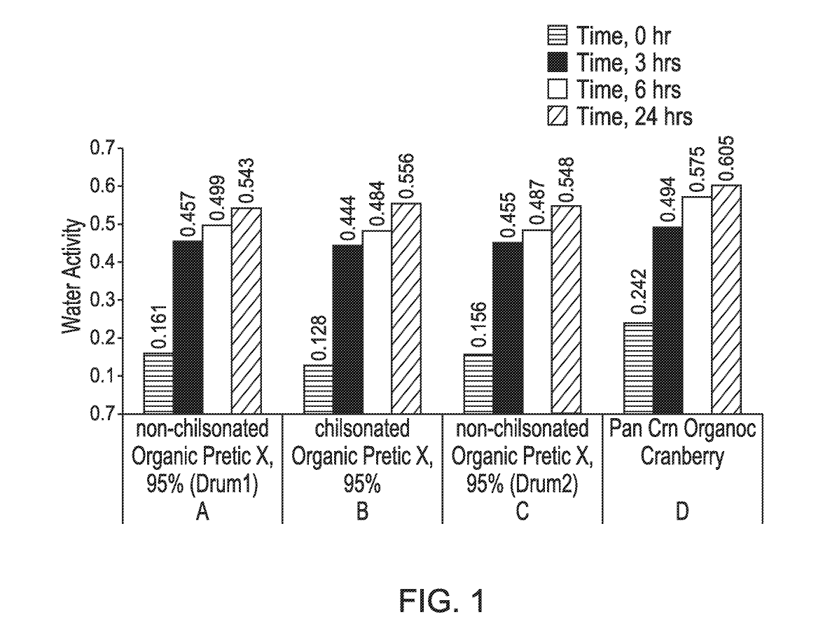

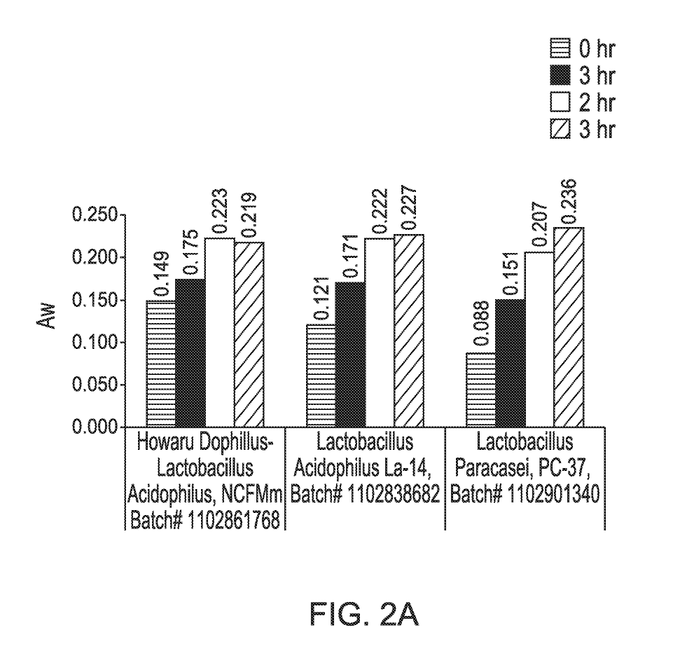

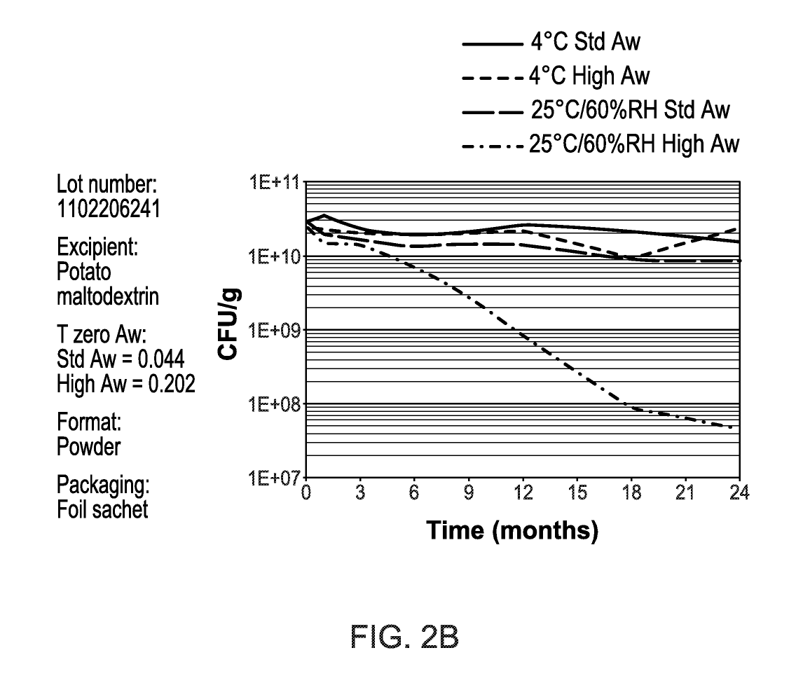

Initially, the process requires selection and blending of ingredients to achieve the lowest possible starting aw. The data from the various formulations detailed below show that achieving an ideal a_{w} is not merely a matter of blending ingredients with low initial a_{w} levels. Virtually all suitable excipients of agricultural origin and of commercial value are very hygroscopic, which means that any exposure to humidity during production, storage, or blending results in rapid increases in water activity (see FIG. 1). Cultures are also highly hygroscopic and rapidly increase in a_{w} during handling (see FIG. 2A). Accordingly, the probiotics and excipients for use in the encapsulated probiotic compositions are initially selected to include a desired initial a_{w} for each and in amounts that will provide the desired potency. Particularly, probiotic strains are selected alone or in combination to have an initial a_{w} of probiotics of less than 0.15 a_{w} and to provide a potency of at least 5 Billion CFU/capsule. The excipients are selected to have an initial a_{w} of less than 0.30 aw, and suitably, from about 0.10 a_{w} to about 0.20 a_{w}, which can be achieved through the chilsonation process described below.

As noted above, however, selection of probiotics and excipients with desired initial a_{w} is not sufficient to ensure that the end encapsulated probiotic composition can be prepared with a water activity to allow for stability and capsule integrity. Accordingly, after determining the types and amounts of probiotics and excipients to form the encapsulated probiotic composition, chilsonation, a known mechanical milling process, has been adapted in the process of the present disclosure in order to improve blending and reduce initial ingredient water activities of the excipients to be used in the prepared encapsulated probiotic compositions of the present disclosure. Normally, chilsonation is a milling treatment that can be used to adjust particle size and bulk density of powdered ingredients. Particularly, chilsonation is a process of dry agglomeration. This treatment was modified by use of specific settings and specific components (e.g., rotors, power settings, gap sizes, screw speeds) to adjust initial excipient water activities. That is, the present disclosure utilizes a range of chilsonation settings that allow for the reduction of water activities over initial levels by up to 25% to 50%, moving initial aw's into a much more favorable range for blending and packaging. Particularly, the chilsonation process is performed on the individual excipients as needed and is used to reduce the water activity (a_{w}) of the individual excipients from about 0.2 a_{w} to about 0.1 a_{w} prior to being blended with the probiotics; that is, the chilsonation process has been adapted herein to be a drying process that does not damage active ingredients.

Once blended, the bulk composition of probiotic and excipient is encapsulated using standard encapsulation methods. As noted above, the capsule is typically made of a plant-derived water soluble polysaccharide, including gums and starches such as, for example, tapioca, acacia, locust bean, and the like, as well as combinations thereof.

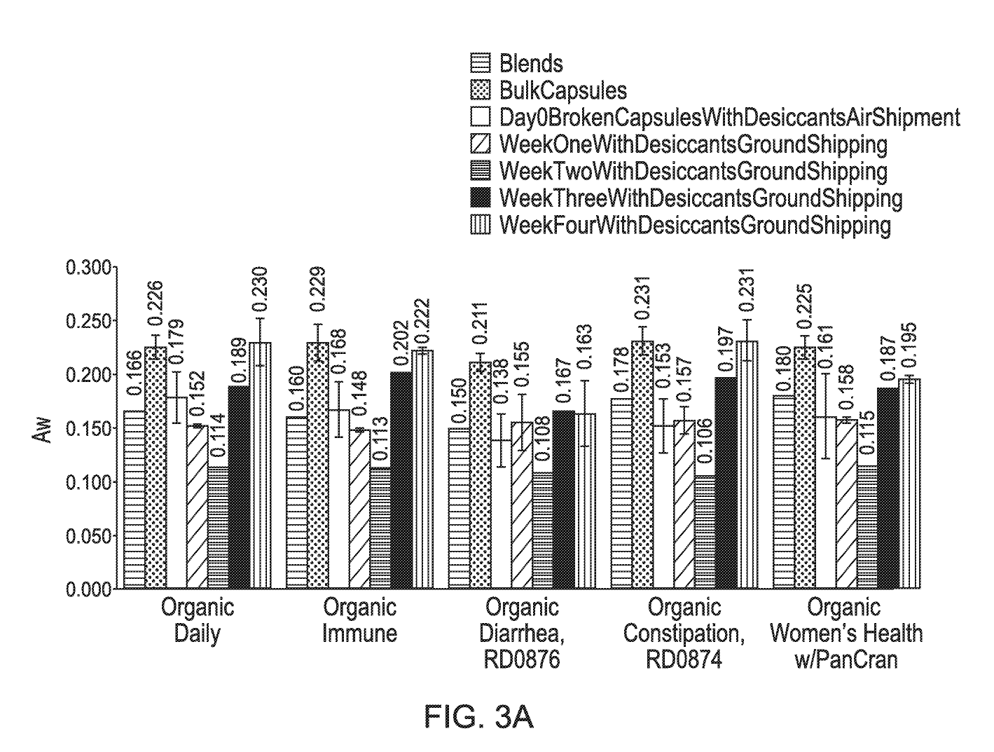

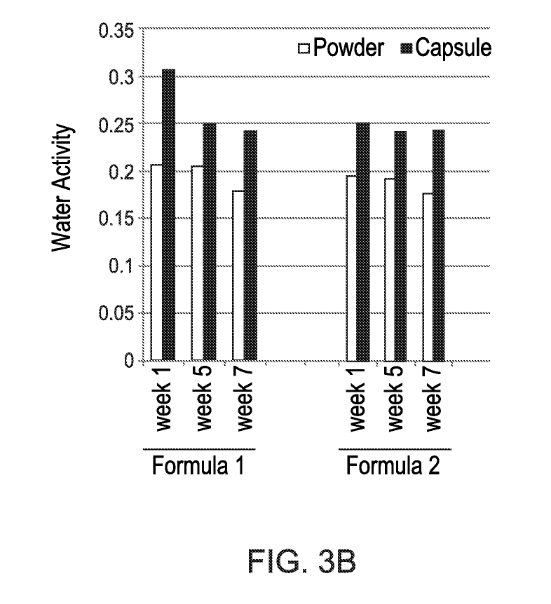

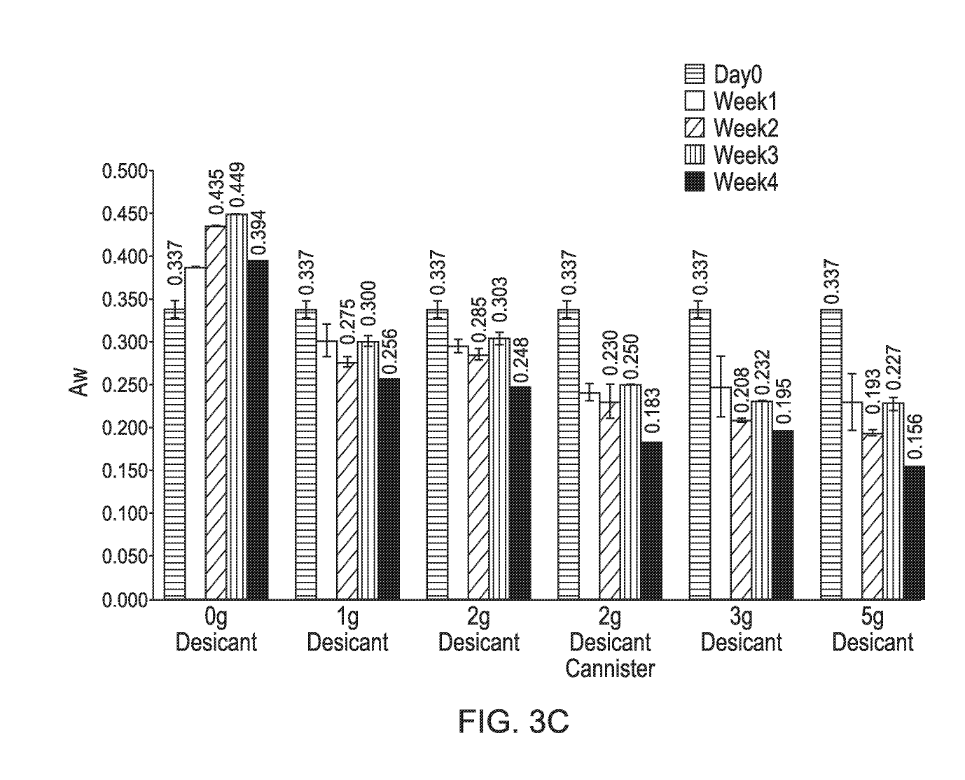

Additionally, even under the best possible blending and processing conditions, it is many times still not possible to ensure a final encapsulated product water activity that meets product needs. For example, the water activity of filled capsules changes in significant and non-intuitive ways during the first month after packaging into bottles. Particularly, with reference to FIG. 3A, a low initial blended a_{w} gives way to higher a_{w} in bulk capsules as free moisture first begins to migrate from capsule to contents (the blend of excipient and probiotic cultures), causing an initial increase in aw. Next, a_{w} enters a phase of decline, as free moisture migrates from the capsules into the desiccant canisters or pillows that are present in the bottles, or in dessicant layered bottles or blister packs. After hitting a low point in ˜2 weeks at 5° C., the a_{w} again begins to rise due to additional migration and equilibration of water, until it reaches an equilibrium level around 4 weeks that will determine the overall longevity and stability of the active culture during shelf life. These changes show that the initial a_{w} is not completely predictive of where an encapsulated probiotic composition will end up for long term aw. The rate and extent of moisture migration during equilibration are key variables for creation of a stable end encapsulated probiotic composition.

Suitably, the encapsulated probiotic compositions of the present disclosure reach a desired water activity in about three to six weeks (equilibration period) and a storage temperature ranging from about 0° C. to about 40° C., suitably from about 4° C. to about 25° C. of 0.2 a_{w} or less (see FIG. 3B).

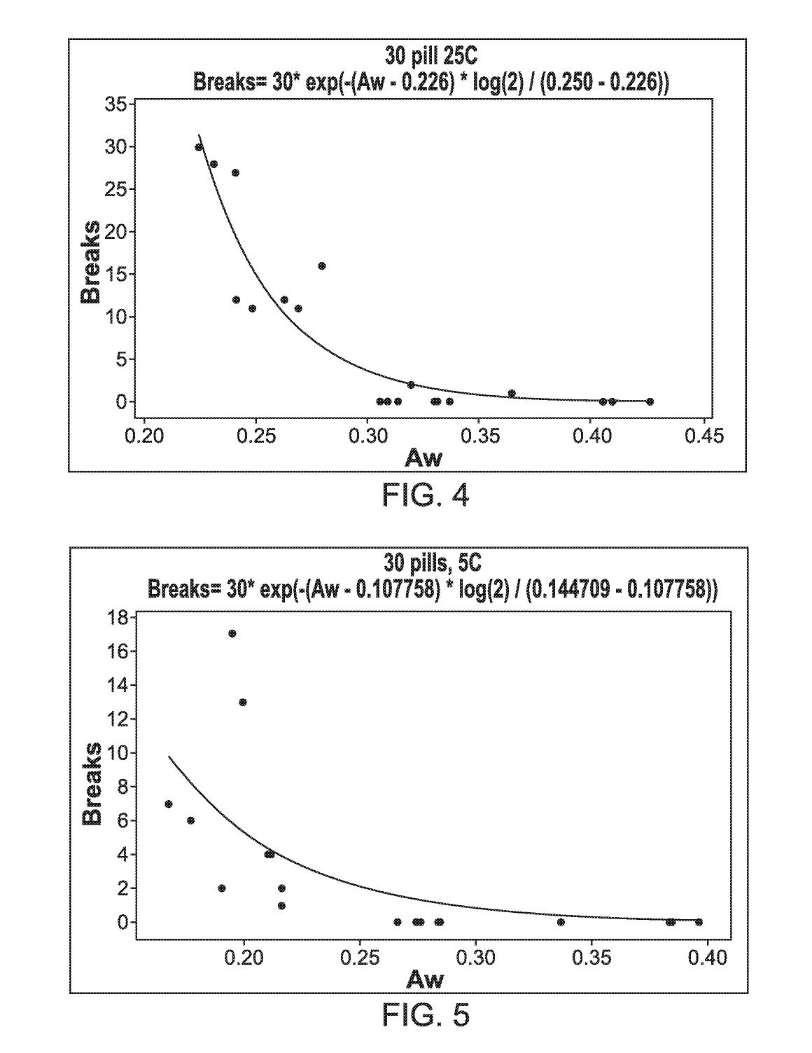

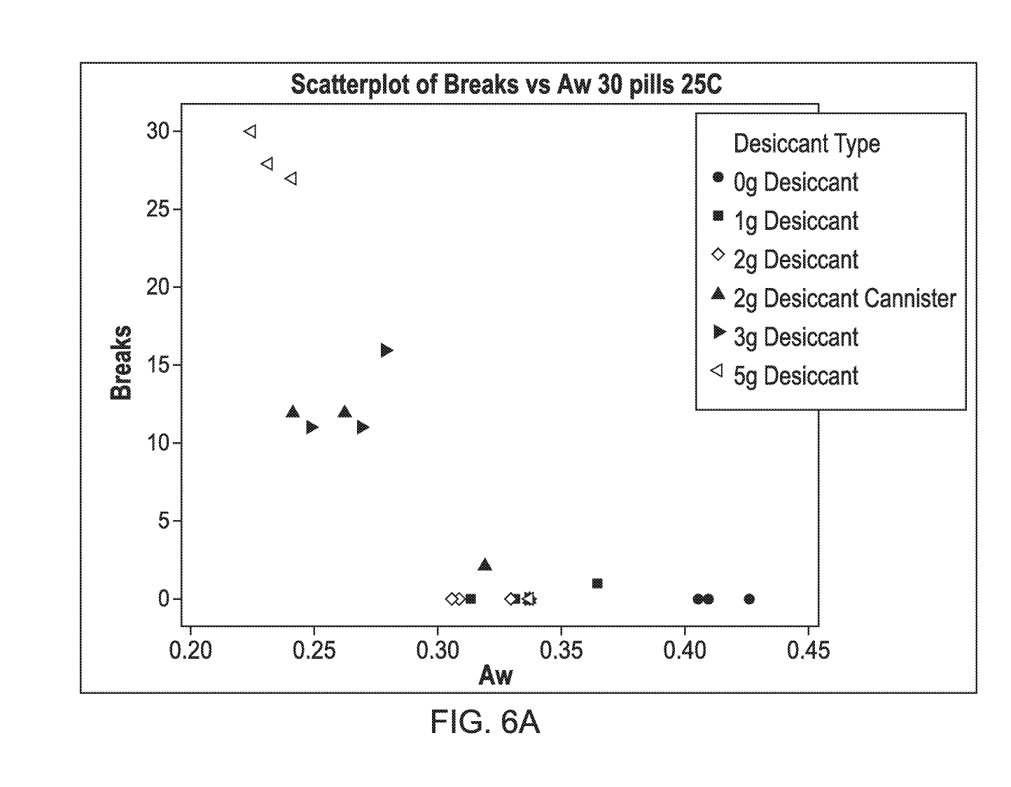

Typically, there is a tradeoff between water activity and breakage when encapsulated probiotic compositions (also referred to as capsules herein) have been conditioned at 25° C. for up to 4 weeks (shown in FIGS. 4 and 5). By drying capsules to different a_{w} levels and then conducting a crush test, it is possible to determine the minimum breakage water activity threshold, defined as the a_{w} at which 50% of capsules will break during a crush test. Particularly, a weight (99.4 grams) is placed inside a hollow portion of a cylinder. This weight is held at the top by a lever. The capsule to be tested is placed on a flat surface and lever and weight assembly is placed above the capsule. Then the lever is released. This enables the weight to travel 4 inches before it hits the capsule. If the capsule is brittle, it causes a breakage. This type of breakage helps determine if the empty/filled capsule is brittle. This enables prediction of whether or not shipping and handling would cause breakage of capsules when shipped and stored under certain conditions.

By conducting crush testing on up to 30 capsules at each a_{w} point, a curve can be derived showing the exact breakage point. In FIG. 4, minimum breakage water activity threshold is calculated based on the curve to be 0.3. So at 0.25 aw, 50% of capsules are expected to fail in the crush test, and would also likely break in the bottle during shipping and storage over time. On the other hand, the minimum breakage water activity threshold for capsules tempered at 5° C. was much lower, approximately 0.2 to 0.23 (FIG. 5). Temperature during the equilibration period turns out to be a critical variable that determines the tendency of capsules to break, and can be manipulated as part of the equilibration process.

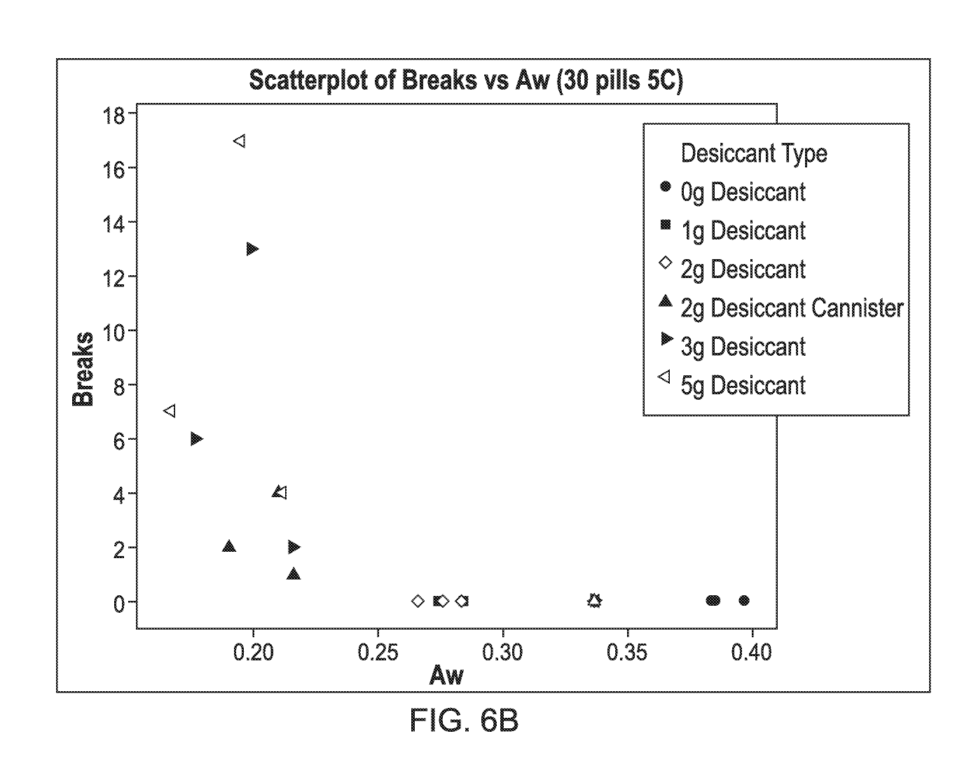

It has been discovered that the degree of capsule breakage can be manipulated by the rate at which moisture is removed from the capsules and the contents. One step involves tailoring the amount and form of desiccant to the overall moisture load in each end encapsulated probiotic composition product (i.e., probiotic capsule). A modelling system is used to identify an ideal desiccant amount versus fill weight for each type of probiotic capsule. FIGS. 6A & 6B show that using high levels of desiccant (e.g., 5 grams) actually increases breakage levels, resulting in uneven declines in moisture, and causing high levels of capsule breakage even at overall high average a_{w} levels (>0.25). On the other hand, the use of very low levels of desiccant fails to remove sufficient moisture (below ˜0.22). In this scenario, capsules can be stable, but culture stability is compromised.

By determining desired levels of desiccant and lowering the equilibration temperature, it becomes possible to generate stable capsules with lower a_{w} levels. Model conditions have now been determined to enable production of commercial encapsulated probiotic compositions based on a range of different excipients, culture types, bottle counts, and packaging types.

FIG. 3C gives an example of how final moisture levels can be adjusted by use of different desiccants and temperatures during the equilibration period. This allows for the production of an encapsulated product composition having a suitable combination of culture stability and capsule integrity.

The present disclosure is further directed to kits including the encapsulated probiotic compositions and containers for packaging the compositions. That is, once prepared, the encapsulated probiotic compositions can be packaged into a container for sale to consumers. Suitable containers include bottles, canisters, blister packs, stick packs (form-fill-seal flexible packaging), and vials, and the like.

The improvement in culture stability supported by the present disclosure allows creation of new higher potency probiotic products that can address a wider range of conditions, many of which require guaranteed potencies above what was previously possible. Higher guaranteed potencies has allowed for the formulation of products containing multiple clinically proven strains and benefits, in effect, allowing production of a multi-vitamin approach to probiotic formulation.

### EXAMPLES

### Exemplary Formulations

The following exemplary encapsulated probiotic composition embodiments are provided solely by way of example and are not intended to limit the scope of the present disclosure in any way. Consistently, various other formulation embodiments of the encapsulated probiotic compositions and methods of manufacture and packaging the same, within the scope of the present disclosure are disclosed herein.

TABLE 1
Exemplary Formula for Treating Constipation

| | | | | | |
|---|---|---|---|---|---|
| | | Total | | Input Qty | |
| Ingredients (per capsule) | Billion CFU/g | CFUs | Potency (%) | (mg) | % of Formula |

| | | | |
|---|---|---|---|
| Organic XOS | 95.0 | 664.50 | 78.2 |

Probiotic Blend

| | | | | | |
|---|---|---|---|---|---|
| B. lactis - strain 1 | 396 | 5.62 | 100.0 | 14.19 | 1.7 |
| B. bifidum/B. lactis | 390 | 1.39 | 100.0 | 3.56 | 0.4 |
| (strain 2) blend | | | | | |
| B. lactis - strain 3 | 458 | 1.21 | 100.0 | 2.64 | 0.3 |
| L. paracasei | 377 | 0.69 | 100.0 | 1.83 | 0.2 |
| L. casei | 318 | 0.61 | 100.0 | 1.92 | 0.2 |
| L. salivarius | 280 | 0.56 | 100.0 | 2.00 | 0.2 |
| L. plantarum | 438 | 0.89 | 100.0 | 2.03 | 0.2 |
| L. acidophilus | 231 | 0.63 | 100.0 | 2.73 | 0.3 |
| L. rhamnosus | 533 | 0.32 | 100.0 | 0.60 | 0.1 |
| B. lactis - strain 4 | 493 | 0.37 | 100.0 | 0.75 | 0.1 |
| Microcrystalline | | | 100.0 | 5.25 | 0.6 |
| cellulose | | | | | |
| Organic NU-FLOW ® | | | 100.0 | 30.00 | 3.5 |
| Organic vegetable | | | | 118.00 | 13.9 |
| capsule | | | | | |

TABLE 2
Exemplary Formula for use as a Daily Supplement

| | | | | | |
|---|---|---|---|---|---|
| | | Total | | Input Qty | |
| Ingredients (per capsule) | Billion CFU/g | CFUs | Potency (%) | (mg) | % of Formula |

| | | | |
|---|---|---|---|
| Organic XOS | 95.0 | 664.52 | 78.2 |

Probiotic Blend

| | | | | | |
|---|---|---|---|---|---|
| L. rhamnosus | 526 | 3.80 | 100.0 | 7.23 | 0.9 |
| B. lactis - strain 1 | 396 | 2.81 | 100.0 | 7.09 | 0.8 |
| B. lactis - strain 2 | 492 | 1.47 | 100.0 | 2.99 | 0.4 |
| B. lactis - strain 3 | 460 | 0.81 | 100.0 | 1.76 | 0.2 |
| B. bifidum/B. lactis | 394 | 0.56 | 100.0 | 1.42 | 0.2 |
| (strain 4) blend | | | | | |
| L. paracasei | 377 | 0.69 | 100.0 | 1.83 | 0.2 |
| L. casei | 318 | 0.61 | 100.0 | 1.92 | 0.2 |
| L. salivarius | 280 | 0.56 | 100.0 | 2.00 | 0.2 |
| L. plantarum | 438 | 0.89 | 100.0 | 2.03 | 0.2 |
| L. acidophilus | 231 | 0.63 | 100.0 | 2.73 | 0.3 |
| Microcrystalline | | | 100.0 | 6.48 | 0.8 |
| cellulose | | | | | |
| Organic NU-FLOW ® | | | 100.0 | 30.00 | 3.5 |
| Organic vegetable | | | | 118.00 | 13.9 |
| capsule | | | | | |

TABLE 3
Exemplary Formula for Treating Diarrhea

| | | | | | |
|---|---|---|---|---|---|
| | | Total | | Input Qty | |
| Ingredients (per capsule) | Billion CFU/g | CFUs | Potency (%) | (mg) | % of Formula |

| | | | |
|---|---|---|---|
| Organic XOS | 95.0 | 664.51 | 78.2 |

Probiotic Blend

| | | | | | |
|---|---|---|---|---|---|
| B. lactis - strain 1 | 396 | 2.81 | 100.0 | 7.09 | 0.8 |
| B. bifidum/B. lactis | 391 | 2.56 | 100.0 | 6.54 | 0.8 |
| (strain 2) blend | | | | | |
| B. lactis - strain 3 | 492 | 1.84 | 100.0 | 3.74 | 0.4 |
| B. lactis - strain 4 | 459 | 2.02 | 100.0 | 4.40 | 0.5 |
| L. paracasei | 381 | 1.74 | 100.0 | 4.57 | 0.5 |
| L. acidophilus - strain 1 | 231 | 1.52 | 100.0 | 6.58 | 0.8 |
| L. casei | 316 | 0.06 | 100.0 | 0.19 | 0.0 |
| L. salivarius | 300 | 0.06 | 100.0 | 0.20 | 0.0 |
| L. plantarum | 450 | 0.09 | 100.0 | 0.20 | 0.0 |
| L. acidophilus - strain 2 | 222 | 0.06 | 100.0 | 0.27 | 0.0 |
| Microcrystalline | | | 100.0 | 3.71 | 0.4 |
| cellulose | | | | | |
| Organic NU-FLOW ® | | | 100.0 | 30.00 | 3.5 |
| Organic vegetable | | | | 118.00 | 13.9 |
| capsule | | | | | |

TABLE 4
Exemplary Formula to Boost and/or Support Immune Function

| | | | | | |
|---|---|---|---|---|---|
| | | Total | | Input Qty | |
| Ingredients (per capsule) | Billion CFU/g | CFUs | Potency (%) | (mg) | % of Formula |

| | | | |
|---|---|---|---|
| Organic XOS | 95.0 | 664.51 | 78.2 |

Probiotic Blend

| | | | | | |
|---|---|---|---|---|---|
| B. lactis - strain 1 | 396 | 2.81 | 100.0 | 7.09 | 0.8 |
| B. bifidum/B. lactis | 391 | 2.50 | 100.0 | 6.40 | 0.8 |
| (strain 2) blend | | | | | |
| B. lactis - strain 3 | 492 | 2.76 | 100.0 | 5.61 | 0.7 |
| L. paracasei | 381 | 1.74 | 100.0 | 4.57 | 0.5 |
| L. acidophilus - strain 1 | 231 | 0.76 | 100.0 | 3.29 | 0.4 |
| B. lactis - strain 4 | 460 | 0.81 | 100.0 | 1.76 | 0.2 |
| L. casei | 313 | 0.30 | 100.0 | 0.96 | 0.1 |
| L. salivarius | 280 | 0.28 | 100.0 | 1.00 | 0.1 |
| L. plantarum | 446 | 0.45 | 100.0 | 1.01 | 0.1 |
| L. acidophilus - strain 2 | 234 | 0.32 | 100.0 | 1.37 | 0.2 |
| Microcrystalline | | | 100.0 | 4.43 | 0.5 |
| cellulose | | | | | |
| Organic NU-FLOW ® | | | 100.0 | 30.00 | 3.5 |
| Organic vegetable | | | | 118.00 | 13.9 |
| capsule | | | | | |

TABLE 5
Exemplary Formula to Boost and/or Support Immune Function for Children

| | | | | | |
|---|---|---|---|---|---|
| | | Total | | Input Qty | |
| Ingredients (per capsule) | Billion CFU/g | CFUs | Potency (%) | (mg) | % of Formula |

| | | | |
|---|---|---|---|
| Organic XOS | 95.0 | 1500.00 | 92.8 |

Probiotic Blend

| | | | | | |
|---|---|---|---|---|---|
| B. lactis - strain 1 | 492 | 5.51 | 100.0 | 11.21 | 0.7 |
| B. lactis - strain 2 | 251 | 2.81 | 100.0 | 7.09 | 0.4 |
| L. paracasei | 310 | 3.47 | 100.0 | 9.14 | 0.6 |
| L. acidophilus - strain 1 | 136 | 1.52 | 100.0 | 6.58 | 0.4 |
| B. lactis - strain 3 | 144 | 1.61 | 100.0 | 3.52 | 0.2 |
| B. bifidum/B. lactis | 10 | 0.11 | 100.0 | 0.28 | 0.0 |
| (strain 4) blend | | | | | |
| L. plantarum | 16 | 0.18 | 100.0 | 0.41 | 0.0 |
| L. salivarius | 10 | 0.11 | 100.0 | 0.40 | 0.0 |
| L. acidophilus - strain 2 | 12 | 0.13 | 100.0 | 0.55 | 0.0 |
| L. casei | 11 | 0.12 | 100.0 | 0.38 | 0.0 |
| Microcrystalline | | | 100.0 | 10.43 | 0.6 |
| cellulose | | | | | |
| Organic NU-FLOW ® | | | 100.0 | 30.00 | 1.9 |

TABLE 6
Exemplary Formula for Women's Health

| | | Total | | Input Qty | |
|---|---|---|---|---|---|
| Ingredients (per capsule) | Billion CFU/g | CFUs | Potency (%) | (mg) | % of Formula |
| Organic Whole | | | 100.0 | 260.00 | 30.6 |
| Cranberry Extract | | | | | |
| Organic XOS | | | 95.0 | 369.52 | 43.5 |

Probiotic Blend

| | | | | | |
|---|---|---|---|---|---|
| L. rhamnosus | 525 | 4.49 | 100.0 | 8.56 | 1.0 |
| B. lactis - strain 1 | 396 | 2.81 | 100.0 | 7.09 | 0.8 |
| L. acidophilus | 231 | 2.53 | 100.0 | 10.93 | 1.3 |
| B. lactis - strain 2 | 492 | 1.47 | 100.0 | 2.99 | 0.4 |
| B. lactis - strain 3 | 460 | 0.81 | 100.0 | 1.76 | 0.2 |
| L. gasseri | 260 | 0.45 | 100.0 | 1.73 | 0.2 |
| L. paracasei | 389 | 0.07 | 100.0 | 0.18 | 0.0 |
| L. casei | 316 | 0.06 | 100.0 | 0.19 | 0.0 |
| L. salivarius | 300 | 0.06 | 100.0 | 0.2 | 0.0 |
| L. plantarum | 450 | 0.09 | 100.0 | 0.2 | 0.0 |
| Microcrystalline | | | 100.0 | 3.65 | 0.4 |
| cellulose | | | | | |
| Organic NU-FLOW ® | | | 100.0 | 65.00 | 7.6 |
| Organic vegetable | | | | 118.00 | 13.9 |
| capsule | | | | | |

What is claimed is:

1. An encapsulated probiotic composition comprising a capsule and 5% or less by weight probiotic, wherein the probiotic composition has a potency of at least 5 Billion CFU/capsule through the end of shelf-life.

2. The encapsulated probiotic composition as set forth in claim 1, wherein the capsule is an organic capsule.

3. The encapsulated probiotic composition as set forth in claim 2, wherein the capsule comprises a plant-derived water soluble polysaccharide.

4. The encapsulated probiotic composition as set forth in claim 1 having a potency ranging from about 5 Billion CFU/capsule to about 30 Billion CFU/capsule through the end of shelf-life.

5. The encapsulated probiotic composition as set forth in claim 1 having a potency ranging from about 5 Billion CFU/capsule to about 10 Billion CFU/capsule through the end of shelf-life.

6. The encapsulated probiotic composition as set forth in claim 1, wherein the capsule comprises an initial water activity (a_{w}) at a temperature of from about 4° C. to about 37° C. ranging from about 0.20 to less than 0.60.

7. The encapsulated probiotic composition as set forth in claim 1 further comprising at least one excipient.

8. The encapsulated probiotic composition as set forth in claim 7, wherein the at least one excipient has an initial water activity (a_{w}) of less than 0.30.

9. The encapsulated probiotic composition as set forth in claim 7, wherein the at least one excipient is selected from the group consisting of prebiotic oligosaccharides, prebiotic fibers, and combinations thereof.

10. The encapsulated probiotic composition as set forth in claim 9, wherein the at least one excipient is selected from the group consisting of organic xylo-oligosaccharide (XOS), fructo-oligosaccharide (FOS), Inulin (fiber), aranbinoxylan (fiber), and combinations thereof.

11. The encapsulated probiotic composition as set forth in claim 7, wherein the at least one excipient is selected from dried fungal fermentates, yeasts, whole fuits, berries, botanicals, extracts, betaglucan, cereals, cellulose and combinations thereof.

12. An encapsulated probiotic composition comprising a capsule and 5% or less by weight probiotic, wherein the probiotic composition has an initial water activity (a_{w}) less than 0.60.

13. The encapsulated probiotic composition as set forth in claim 12, wherein the capsule comprises a water activity (a_{w}) at a temperature of from about 4° C. to about 37° C. ranging from 0.20 to less than 0.60.

14. The encapsulated probiotic composition as set forth in claim 12, wherein the capsule comprises an initial water activity (a_{w}) at a temperature of from about 4° C. to about 25° C. ranging from about 0.20 and to less than 6.0.

15. The encapsulated probiotic composition as set forth in claim 12, wherein the capsule is an organic capsule.

16. The encapsulated probiotic composition as set forth in claim 12 having a potency of at least 5 Billion CFU/capsule.

17. The encapsulated probiotic composition as set forth in claim 12 having a potency ranging from about 5 Billion CFU/capsule to about 20 Billion CFU/capsule.

18. The encapsulated probiotic composition as set forth in claim 12 further comprising at least one excipient.

19. A kit comprising a container and the encapsulated probiotic composition of claim 1.

20. A kit comprising a container and the encapsulated probiotic composition of claim 12.
